(19) United States
(12) Patent Application Publication
(10) Pub. No.: US 2009/0136417 A1
(43) Pub. Date: May 28, 2009

(54) GLYCEROL ESTER DERIVATIVE HAVING METAL CHELATE STRUCTURE

(75) Inventors: Kazunobu Takahashi, Kanagawa (JP); Kazuhiro Aikawa, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: Fujifilm Corporation, Minato-ku Tokyo (JP)

(21) Appl. No.: 11/910,190

(22) PCT Filed: Mar. 31, 2006
(86) PCT No.: PCT/JP2006/307383

§ 371 Date: Nov. 12, 2007

(30) Foreign Application Priority Data
Apr. 1, 2005 (JP) 2005 105800

(51) Int. Cl. A61K 51/12; C07C 69/34; C07F 9/09; A61K 49/18

(52) U.S. Cl. 424/1.21; 560/195; 556/26; 424/9.3

(57) ABSTRACT

A compound represented by the following general formula (I):

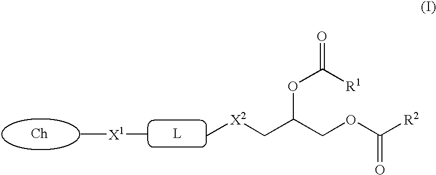

wherein R¹ and R² represent an alkyl group or alkenyl group; X¹ and X² represent —O—, or —N(Z)-; Z represents hydrogen atom, or an alkyl group; L represents a divalent bridging group comprising an alkylene group, an alkenylene group, —CO—, —O— and the like wherein number of atoms constituting a chain length of the bridging group is 1 to 10; and Ch represents a partial structure containing 3 or more nitrogen atoms and capable of forming a chelate, and a chelate compound comprising the compound are provided. A vascular lesion can be selectively contrasted by MRI or scintigraphy imaging using a contrast medium comprising liposomes containing said compound or said chelate.

### FIELD OF THE INVENTION

The present invention relates to a glycerol ester derivative or salt thereof which can form a metal chelate structure, and a chelate compound or salt thereof containing said derivative. The present invention further relates to a liposome which contains said derivative or salt thereof, or said chelate compound or salt thereof as a membrane component, and a contrast medium which comprises said liposome.

### BACKGROUND ART

A primary example of non-invasive methods for diagnosing arteriosclerosis includes X-ray angiography. This method involves contrasting vascular flows by using a water-soluble iodine-containing contrast medium, and for this reason, the method has a problem of poor distinguishability of pathological tissues from normal tissues. The above method only achieves detection of a lesion where constriction progresses 50% or more, and the method has difficulty in detecting a lesion before onset of attack of an ischemic disease.

As diagnostic methods other than the above, methods of detecting a disease by nuclear magnetic resonance tomography (MRI) have been reported in recent years which use a contrast medium kinetically distributed at a high level in arteriosclerotic plaques. However, any of compounds reported as the contrast medium have a problem when used in diagnostic methods. For example, hematoporphyrin derivatives are reported to have a defect of deposition in the skin and coloring of the skin (see, U.S. Pat. No. 4,577,636). Gadolinium complexes having a perfluorinated side chain, which have been reported to accumulate in lipid-rich plaques, are concerned to accumulate in lipid-rich tissues and organs in vivo, such as fatty livers, renal epitheliums, and tendons of muscular tissues (see, Circulation, 109, 2890, 2004).

From a viewpoint of chemical compounds, compounds comprising phosphatidylethanolamine (PE) and diethylenetriaminepentaacetic acid (DTPA) bonded via an amide bond are known (for example, see, Polymeric Materials Science and Engineering, 89, 148, 2003), and liposomes using gadolinium complexes of such compounds have also been reported (Inorganica ChimicaActa, 331, 151, 2002). However, these complexes are hardly soluble, and therefore, they have a problem of poor maneuverbility in liposome formation, and they are also concerned to be accumulated or toxic in vivo.

### DISCLOSURE OF THE INVENTION

An object of the present invention is to provide a compound suitable for a lesion-selective liposome contrast medium and having superior solubility, and also to provide a contrast medium for MRI and contrast medium for scintigraphy comprising such a compound.

The inventors of the present invention conducted various researches to achieve the aforementioned object. As a result, they found that a glycerol ester derivative having a metal chelate structure had excellent solubility and superior properties as a component of liposomes as a contrast medium for MRI. The present invention was achieved on the basis of these findings.

The present invention thus provides a compound represented by the following general formula (I), or a salt thereof:

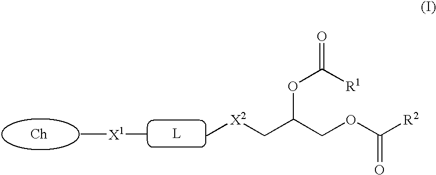

wherein R¹ and R² independently represent an alkyl group or alkenyl group having 8 to 30 carbon atoms; X¹ and X² independently represent —O—, or —N(Z)-; Z represents hydrogen atom, or an alkyl group having 1 to 5 carbon atoms; L represents a divalent bridging group comprising one or more partial structures selected from the group consisting of an alkylene group, an alkenylene group, —CO—, —O—, —NH—, —N═, —S—, —SO—, and —SO₂, wherein number of atoms that constitute a chain length of the bridging group is 1 to 10, and wherein said bridging group may be substituted; and Ch represents a partial structure containing 3 or more nitrogen atoms and capable of forming a chelate, which may be substituted with a substituent selected from the following substituent group (substituent group: hydroxyl group; an alkoxyl group; carboxyl group; carbamoyl group; amino group; an alkylamino group; sulfo group; an alkyl group having 1 to 5 carbon atoms which may be substituted with a substituent selected from the group consisting of hydroxyl group, an alkoxyl group, carboxyl group, carbamoyl group, amino group, an alkylamino group, and sulfo group; and a combination of these groups, provided that a total number of carbon atom, oxygen atom, nitrogen atom, and sulfur atom in each substituent in the substituent group is 1 to 20).

As preferred embodiments of the present invention, there are provided the aforementioned compound or a salt thereof, wherein Ch is represented by the following general formula (II):

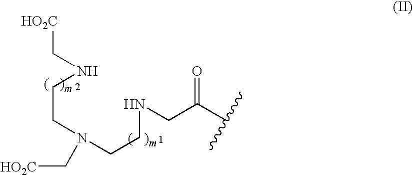

wherein m¹ and m² independently represent an integer of 1 or 2; the aforementioned compound or a salt thereof, wherein Ch is represented by the following formula (III):

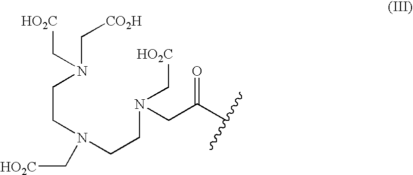

the aforementioned compound or a salt thereof, wherein Ch is represented by the following formula (IV):

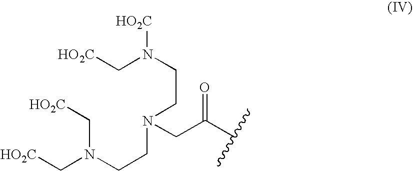

and the aforementioned compound or a salt thereof, wherein Ch is represented by the following general formula (V):

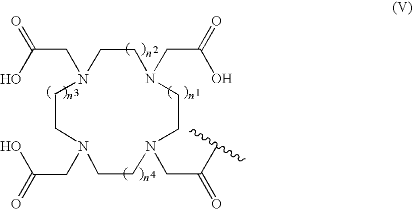

wherein n¹, n², n³, and n⁴ independently represent an integer of 1 or 2.

As other preferred embodiments of the present invention, there are provided the aforementioned compound or a salt thereof, wherein R¹ and R² independently represent an alkyl group having 8 to 30 carbon atoms; the aforementioned compound or a salt thereof, wherein R¹ and R² independently represent an alkenyl group having 8 to 30 carbon atoms; the aforementioned compound or a salt thereof, wherein R¹ and R² independently represent an alkyl group or alkenyl group having 10 to 20 carbon atoms; and the aforementioned compound or a salt thereof, wherein L is a divalent bridging group comprising one or more partial structures selected from the group consisting of an alkylene group, an alkenylene group, —CO—, and —O— and wherein the number of atoms that constitute the chain length of the bridging group is 1 to 10.

As other preferred embodiments of the present invention, there are provided a chelate compound or a salt thereof, which consists of any one of the aforementioned compounds and a metal ion; the aforementioned chelate compound or a salt thereof, wherein the metal ion is derived from an element selected from elements of atomic numbers 21 to 29, 31, 32, 37 to 39, 42 to 44, 49, and 57 to 83; and the aforementioned chelate compound or a salt thereof, wherein the metal ion is derived from a paramagnetic element selected from elements of atomic numbers 21 to 29, 42, 44, and 57 to 71.

From another aspect of the present invention, the present invention provides a liposome containing the aforementioned compound or a salt thereof as a membrane component, and according to a preferred embodiment thereof, the liposome containing a phosphatidylcholine and a phosphatidylserine as membrane components is provided.

From a still further aspect of the present invention, there is provided a contrast medium for MRI comprising the aforementioned liposome. According to preferred embodiments of the above invention, there are provided the aforementioned contrast medium for MRI, which is used for imaging a vascular disease; the aforementioned contrast medium for MRI, which is used for imaging of vascular smooth muscle cells abnormally proliferating under influence of foam macrophages; the aforementioned contrast medium for MRI, which is used for imaging of a tissue or lesion in which macrophages localize; the aforementioned contrast medium for MRI, wherein the tissue in which macrophages localize is selected from the group consisting of tissues of liver, spleen, air vesicle, lymph node, lymph vessel, and renal epithelium; and the aforementioned contrast medium for MRI, wherein the lesion in which macrophages localize is selected from the group consisting of lesions of tumor, inflammation, and infection.

Similarly, the present invention also provides a contrast medium for scintigraphy comprising the aforementioned liposome. According to preferred embodiments of the above invention, there are provided the aforementioned contrast medium for scintigraphy, which is used for imaging a vascular disease; the aforementioned contrast medium for scintigraphy, which is used for imaging of vascular smooth muscle cells abnormally proliferating under influence of foam macrophages; the aforementioned contrast medium for scintigraphy, which is used for imaging of a tissue or lesion in which macrophages localize; the aforementioned contrast medium for scintigraphy, wherein the tissue in which macrophages localize is selected from the group consisting of tissues of liver, spleen, air vesicle, lymph node, lymph vessel, and renal epithelium; and the aforementioned contrast medium for scintigraphy, wherein the lesion in which macrophages localize is selected from the group consisting of lesions of tumor, inflammation, and infection.

Furthermore, the present invention provides use of the aforementioned compound or a salt thereof for the manufacture of the aforementioned contrast medium for MRI or the aforementioned contrast medium for scintigraphy; a method for MRI or a method for scintigraphy imaging, which comprises the step of administering liposomes containing the aforementioned compound as a membrane component to a mammal including human; and a method for imaging a lesion of a vascular disease, which comprises the step of administering liposomes containing the aforementioned compound as a membrane component to a mammal including human.

### BEST MODE FOR CARRYING OUT THE INVENTION

When a functional group is referred to as “which may be substituted” or “which may have a substituent” in the specification, it is meant that the functional group may have one or more substituents. Unless otherwise specifically mentioned, number, substituting position, and type of a substituent to be bound are not particularly limited. When a functional group has two or more substituents, they may be the same or different. In the specification, the “alkyl group” include straight, branched, cyclic alkyl groups, and an alkyl group consisting of a combination thereof, and the cyclic alkyl group include a polycyclic alkyl group such as a bicycloalkyl group. Alkyl moieties of substituents containing the alkyl moieties have the same meaning. In the specification, the “alkenyl group” include straight, branched, cyclic alkenyl groups, and an alkenyl group consisting of a combination thereof, and said group also include a cycloalkenyl group, and a bicycloalkenyl group.

The “partial structure containing 3 or more nitrogen atoms and capable of forming a chelate” in the definition of “Ch” may be a straight, branched, or cyclic structure, or a structure consisting of a combination thereof. Examples of the partial structure include diethylenetriamine unit, 1,4,7,10-tetraazacyclododecane unit, 1,4,8,11-tetraazacyclotetradecane unit, and the like, but not limited to these examples.

In the “Ch”, the number of the substituents, which substitute on the partial structure containing three or more nitrogen atoms and capable of forming a chelate, is generally preferred to be 20 or less, more preferably 10 or less, most preferably 5 or less. Although the substitution position is not particularly limited, substitution on a nitrogen atom is preferred. It is preferred that all of the nitrogen atoms have one or two substituents.

In the group of substituents which substitute on the partial structure containing three or more nitrogen atoms and capable of forming a chelate, the “combination of these groups” means, for example, a functional group comprising an amino group-substituted alkyl group having 1 to 5 carbon atoms of which amino group is substituted with a carboxyl-substituted alkyl group having 1 to 5 carbon atoms, and the like. As for the type of the substituent substituting on the partial structure containing three or more nitrogen atoms and capable of forming a chelate as the “Ch”, a substituted alkyl group having 1 to 5 carbon atoms is preferred, and an alkyl group having 1 to 5 carbon atoms and substituted with carboxyl group, hydroxyl group, or amino group is more preferred. Preferred examples of Ch include diethylenetriaminepentaacetic acid [DTPA] unit, 1,4,7,10-tetraazacyclododecane-1,4,7,10-tetraacetic acid [DOTA] unit, 1,4,8,11-tetraazacyclotetradecane-1,4,8,11-tetraacetic acid [TETA] unit, and the like.

It is also preferred that “Ch” is represented by the following general formula (II).

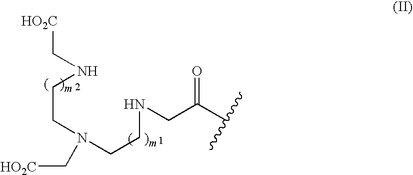

In the general formula (II), m¹ and m² independently represent an integer of 1 or 2, and they each represent a number of methylene in each straight alkylene in the general formula (II). It is more preferred that m¹ and m² both represent 1.

Furthermore, it is also preferred that Ch has a structure represented by the following formula (III):

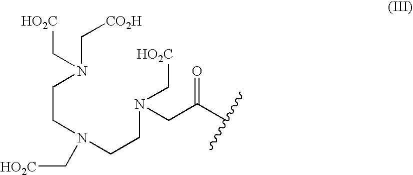

the following formula (IV):

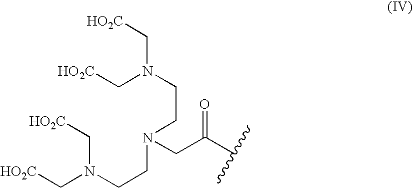

or the following general formula (V):

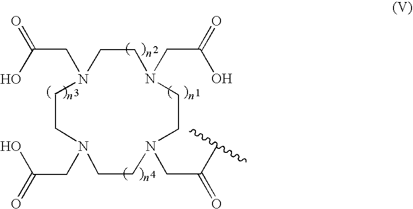

In the general formula (V), n¹, n², n³, and n⁴ independently represent an integer of 1 or 2, and they each represent a number of methylene in each straight alkylene in the general formula (V). It is more preferred that all of n¹, n², n³, and n⁴ represent 1, or n¹ and n³ represent 1, and n² and n⁴ represent 2.

R¹ and R² independently represent an alkyl group or alkenyl group having 8 to 30 carbon atoms. The alkyl group or alkenyl group is preferably a straight or branched alkyl group or alkenyl group, more preferably a straight alkyl group or alkenyl group. Although the alkyl group or alkenyl group may have a substituent, an unsubstituted alkyl group or alkenyl group is preferred. Examples of the substituent include a halogen atom (any of fluorine, chlorine, bromine, and iodine), an alkyl group, an alkenyl group, an alkynyl group, an aryl group, a heterocyclic group, cyano group, hydroxyl group, nitro group, carboxyl group, an alkoxyl group, an aryloxy group, a silyloxy group, a heterocyclyloxy group, an acyloxy group, carbamoyloxy group, an alkoxycarbonyloxy group, an aryloxycarbonyloxy group, amino group (including anilino group), an acylamino group, aminocarbonylamino group, an alkoxycarbonylamino group, an aryloxycarbonylamino group, sulfamoylamino group, an alkyl- or arylsulfonylamino group, mercapto group, an alkylthio group, an arylthio group, a heterocyclylthio group, sulfamoyl group, sulfo group, an alkyl- or arylsulfinyl group, an alkyl- or arylsulfonyl group, an acyl group, an aryloxycarbonyl group, an alkoxycarbonyl group, carbamoyl group, an aryl- or heterocyclylazo group, imido group, phosphino group, phosphinyl group, phosphinyloxy group, phosphinylamino group, and silyl group.

In the alkenyl group represented by R¹ or R², one or more existing double bonds are preferably in the Z-configuration, although the configuration is not particularly limited to this configuration. The position and number of the double bond are not particularly limited. The number is generally preferred to be 5 or less, more preferably 3 or less.

The number of carbon atoms constituting R¹ or R² is more preferably 8 to 25, most preferably 10 to 20. It is preferred that R¹ and R² independently represent an alkyl group having 8 to 30 carbon atoms, and it is similarly preferred that they independently represent an alkenyl group having 8 to 30 carbon atoms.

X¹ and X² independently represent —O—, or —N(Z)-. Z represents hydrogen atom or an alkyl group having 1 to 5 carbon atoms. As Z, an alkyl group having 1 to 3 carbon atoms is more preferred. As X¹ and X², —O—, —NH—, or —NCH₃— is independently most preferred.

The divalent bridging group represented by L may be any of straight, branched and cyclic groups, or a group consisting of a combination thereof. A straight or branched group is preferred, and a straight group is most preferred. Further, the bridging group may be a saturated group, or may contain an unsaturated bond. When the group contains an unsaturated bond, the type, position, and number thereof are not particularly limited. The divalent bridging group represented by L may have a substituent such as cyano group, hydroxyl group, nitro group, carboxyl group, an alkoxyl group, an acyloxy group, carbamoyloxy group, an alkoxycarbonyloxy group, amino group, an acylamino group, aminocarbonylamino group, an alkoxycarbonylamino group, sulfamoylamino group, an alkylsulfonylamino group, mercapto group, an alkylthio group, sulfamoyl group, sulfo group, an alkylsulfinyl group, an alkylsulfonyl group, an acyl group, an alkoxycarbonyl group, carbamoyl group, azo group, and imido group. In the “L”, the number of atoms is preferably 1 to 10 including atoms constituting any substituents other than hydrogen.

Preferred examples of L include a divalent bridging group containing one or more partial structures selected from the group consisting of an alkylene group, an alkenylene group, —CO—, and —O— wherein the number of atoms that constitute the chain length of the bridging group is 1 to 10. Among them, a bridging group containing an alkylene group, an alkenylene group, —O—CH₂CH₂—O— structure, or —O—CH₂CH₂CH₂—O— structure as a partial structure is more preferred.

Preferred examples of the compounds of the present invention will be mentioned below. However, the compounds of the present invention are not limited to these examples.

| | | |
|---|---|---|
| (1) | 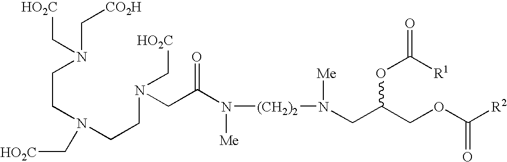 | 1) R¹ = R² = n-C₈H₁₇ 2) R¹ = R² = n-C₁₀H₂₁ 4) R¹ = R² = n-C₁₃H₂₇ 5) R¹ = R² = n-C₁₅H₃₁ 6) R¹ = R² = n-C₁₇H₃₅ 7) R¹ = R² = n-C₂₀H₄₁ 8) R¹ = R² = n-C₂₅H₅₁ 9) R¹ = R² = n-C₃₀H₆₁ |
| (2) | 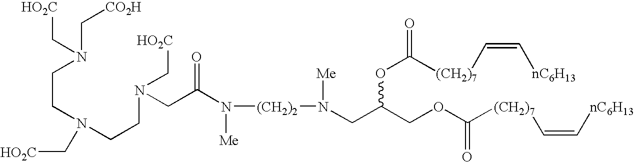 | 1) n = 2 2) n = 4 3) n = 6 4) n = 8 5) n = 10 |
| (3) | 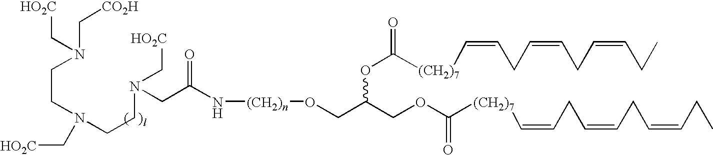 | 1) l = 1, n = 2 2) l = 1, n = 4 3) l = 1, n = 6 4) l = 1, n = 8 5) l = 1, n = 10 6) l = 2, n = 2 7) l = 2, n = 4 8) l = 2, n = 6 9) l = 2, n = 8 10) l = 2, n = 10 |
| (4) | 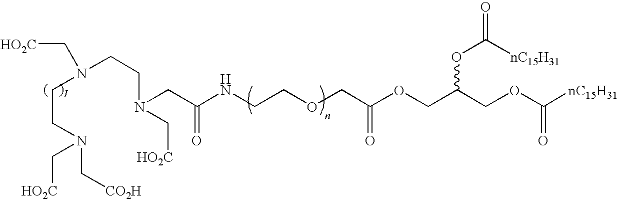 | 1) l = 1, n = 1 2) l = 1, n = 2 3) l = 2, n = 1 4) l = 2, n = 2 |
| (5) | 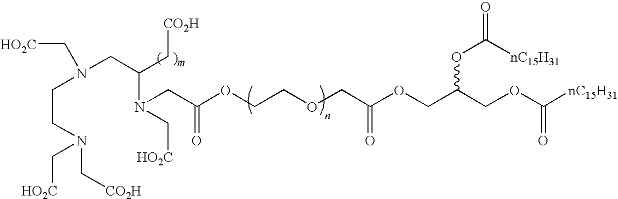 | 1) n = 1, m = 0 2) n = 1, m = 1 3) n = 1, m = 2 4) n = 2, m = 0 5) n = 2, m = 1 6) n = 2, m = 2 |
| (6) | 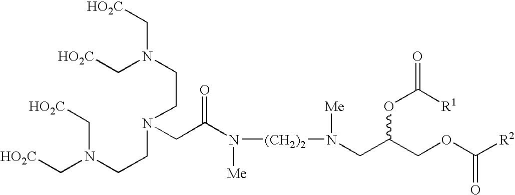 | 1) R¹ = R² = n-C₈H₁₇ 2) R¹ = R² = n-C₁₀H₂₁ 4) R¹ = R² = n-C₁₃H₂₇ 5) R¹ = R² = n-C₁₅H₃₁ 6) R¹ = R² = n-C₁₇H₃₅ 7) R¹ = R² = n-C₂₀H₄₁ 8) R¹ = R² = n-C₂₅H₅₁ 9) R¹ = R² = n-C₃₀H₆₁ |
| (7) | 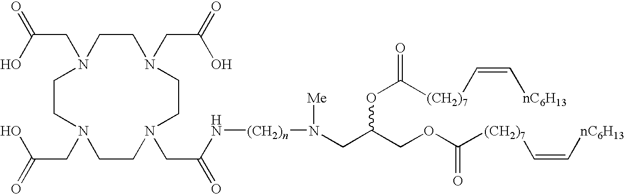 | 1) n = 2 2) n = 4 3) n = 6 4) n = 8 5) n = 10 |
| (8) | 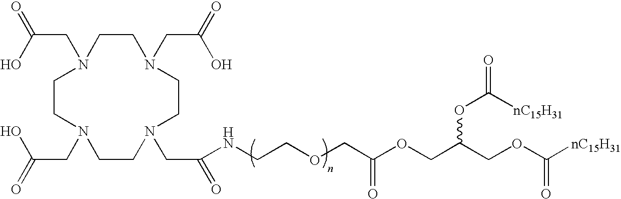 | 1) n = 1 2) n = 2 |
| (9) | 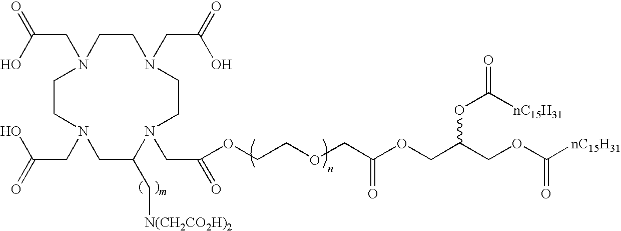 | 1) n = 1, m = 0 2) n = 1, m = 1 3) n = 1, m = 2 4) n = 2, m = 0 5) n = 2, m = 1 6) n = 2, m = 2 |
| (10) | 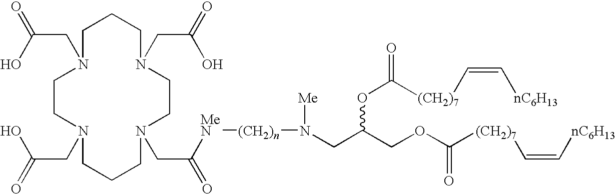 | 1) n = 2 2) n = 4 3) n = 6 4) n = 8 5) n = 10 |

General synthetic methods for the compounds of the present invention will be explained. However, synthetic methods of the compounds of the present invention are not limited to these methods. As the long chain fatty acids as a partial structure of the compounds of the present invention, those ordinarily commercially available may be used, or they may be suitably synthesized depending on purposes. When the compounds are obtained by syntheses, corresponding alcohols and alkyl halides can be used as raw materials according to the method described by Richard C. Larock in Comprehensive Organic Transformations (VCH).

The aforementioned long chain fatty acids can be condensed with a derivative of glycerol, 3-amino-1,2-propanediol, or 1-chloro-2,3-propanediol and thereby derived into a diacyl glyceride derivative. In this process, a protective group can also be used, if necessary. As a protective group used in such a case, for example, any of the protective groups described by T. W. Green & P. G. M. Wuts in “Protecting Groups in Organic Synthesis” (John Wiley & Sons, Inc.) can be suitably selected and used.

The aforementioned diacyl glyceride derivative can be suitably bound with a required bridging group and then bound with a polyamine derivative having a metal coordinating ability to synthesis the compounds of the present invention. This process can be carried out according to the methods described in Bioconjugate Chem., 10, 137 (1999); Tetrahedron Lett., 37, 4685 (1996); J. Chem. Soc., Perkin Trans 2, and 348 (2002); and J. Heterocycl. Chem., 37, 387 (2000). However, these methods are mere examples, and the methods are not limited to these examples.

The chelate compound of the present invention consists of the aforementioned compound and a metal ion. The metal ion is not particularly limited. As metal ions suitable for the purpose of imaging by MRI, X-ray, ultrasonic contrast, scintigraphy, and the like, or radiotherapy, metal ions derived from paramagnetic metals, heavy metals, and radioactive metals of radioactive metal isotopes are preferably used. More specifically, metal ions derived from elements selected from those of the atomic numbers 21 to 29, 31, 32, 37 to 39, 42 to 44, 49, and 57 to 83 are preferred. Examples of metal ions suitable for use of the chelate compounds of the present invention as a contrast medium for MRI include metal ions derived from elements of the atomic numbers 21 to 29, 42, 44 and 57 to 71. For use in the preparation of positive MRI agents, more preferred metals are those of the atomic numbers 24 (Cr), (Mn), 26 (Fe), 63 (Eu), 64 (Gd), 66 (Dy), and 67 (Ho), those of the atomic numbers 25 (Mn), 26 (Fe), and 64 (Gd) are still more preferred, and Mn(II), Fe(III), and Gd(III) are especially preferred. For use in the preparation of negative MRI agents, more preferred metals are those of the atomic numbers 62 (Sm), 65 (Tb), and 66 (Dy).

The compounds and the chelate compounds of the present invention may have one or more asymmetric centers. In such compounds, stereoisomers such as optically active substances and diastereomers based on the asymmetric centers may exist. Any of arbitrary stereoisomers in pure forms, arbitrary mixtures of stereoisomers, racemates and the like fall within the scope of the present invention. Further, the compounds of the present invention may have one or more olefinic double bonds. The configuration thereof may be either E-configuration or Z-configuration, or the compounds may be present as a mixture thereof. The compounds of the present invention may also exist as tautomers. Any tautomers or mixtures thereof fall within the scope of the present invention. Further, the compounds and the chelate compounds of the present invention may form a salt, and the compounds in a free form or the compounds in the form of a salt may form a hydrate or a solvate. All of these substances also fall within the scope of the present invention. The type of the salt is not particularly limited, and the salt may be an acid addition salt, or a base addition salt.

The compounds and the chelate compounds of the present invention, and salts of any of these substances can be used as a membrane component of a liposome. When a liposome is prepared by using the compound or the chelate compound of the present invention or the salt thereof, an amount of the compound or the chelate compound of the present invention, or the salt thereof is generally about from 10 to 90 mass %, preferably from 10 to 80 mass %, further preferably from 20 to 80 mass %, based on the total mass of membrane components. A single kind of the compound or the chelate compound of the present invention or the salt thereof may be used, or two or more kinds may be used in combination.

As other membrane components of liposome, any of lipid compounds ordinarily used for the preparation of liposomes can be used. Such compounds are described in, for example, Biochim. Biophys. Acta, 150 (4), 44 (1982); Adv. in Lipid. Res., 16 (1) 1 (1978); “RESEARCH IN LIPOSOMES”, P. Machy, L. Leserman, John Libbey EUROTEXT Co.); “Liposome” (Ed., Nojima, Sunamoto and Inoue, Nankodo) and the like. As the lipid compounds, phospholipids are preferred, and phosphatidylcholines (PC) are particularly preferred. Preferred examples of phosphatidylcholines include egg PC, dimyristoyl-PC (DMPC), dipalmitoyl-PC (DPPC), distearoyl-PC (DSPC), dioleyl-PC (DOPC) and the like. However, PCs are not limited to these examples.

Preferred examples of membrane component of liposomes include combination of a phosphatidylcholine and a phosphatidylserine (PS). Examples of the phosphatidylserine include those having lipid moieties similar to those of the phospholipids mentioned as preferred examples of the phosphatidylcholines. When a phosphatidylcholine and a phosphatidylserine are used in combination, molar ratio of PC and PS (PC:PS) used is preferably in the range of 90:10 to 10:90

Another preferred embodiment of the liposome of the present invention includes a liposome containing a phosphatidylcholine and a phosphatidylserine and further containing a phosphoric acid dialkyl ester as membrane components. The two alkyl groups constituting the dialkyl ester of phosphoric acid are preferably the same kind of groups, and each alkyl group preferably contains 6 or more carbon atoms, more preferably 10 or more carbon atoms, still more preferably 12 or more carbon atoms. Preferred examples of the phosphoric acid dialkyl ester include, but not limited to, dilauryl phosphate, dimyristyl phosphate, dicetyl phosphate and the like. In the aforementioned embodiment, preferred amount of the phosphoric acid dialkyl ester is from 1 to 50 mass %, preferably from 1 to 30 mass %, further preferably from 1 to 20 mass %, based on the total mass of phosphatidylcholine and phosphatidylserine

In the liposome containing a phosphatidylcholine, a phosphatidylserine, a phosphoric acid dialkyl ester, and the compound or the chelate of the present invention or the salt thereof as membrane components, preferred weight ratios of PC, PS, phosphoric acid dialkyl ester, and the compound or the chelate of the present invention or the salt thereof is from 5 to 40 mass %: from 5 to 40 mass %: from 1 to 10 mass %: from 15 to 80 mass %.

The components of the liposome of the present invention are not limited to the aforementioned four kinds of compounds, and other components may be admixed. Examples of such components include cholesterol, cholesterol esters, sphingomyelin, monosial ganglioside GM1 derivatives described in FEBS Lett., 223, 42 (1987); Proc. Natl. Acad. Sci., USA, 85, 6949 (1988) etc., glucuronic acid derivatives described in Chem. Lett., 2145 (1989); Biochim. Biophys. Acta, 1148, 77 (1992) and the like, polyethylene glycol derivatives described in Biochim. Biophys. Acta, 1029, 91 (1990); FEBS Lett., 268, 235 (1990) and the like. However, the components are not limited to these examples.

The liposomes of the present invention can be prepared by any methods available in this field. Examples of the preparation methods are described in Ann. Rev. Biophys. Bioeng., 9, 467 (1980), “Liopsomes” (Ed. by M. J. Ostro, MARCELL DEKKER, INC.) and the like, as well as the published reviews of liposomes mentioned above. More specifically, examples include the ultrasonication method, ethanol injection method, French press method, ether injection method, cholic acid method, calcium fusion method, freeze and thawing method, reverse phase evaporation method and the like. However, the preparation methods are not limited to these examples. Size of the liposome may be any of those obtainable by the aforementioned methods. Generally, the size in average may be 400 nm or less, preferably 200 nm or less. Structure of the liposome is not also particularly limited, and may be any structure such as unilamellar or multilamellar structure. It is also possible to add one or more kinds of appropriate medicaments or other contrast media inside the liposomes.

When the liposomes of the present invention are used as a contrast medium, they can be preferably administered parenterally, more preferably administered intravenously. For example, preparations in the form of an injection or a drip infusion can be provided as powdery compositions in a lyophilized form, and they can be used by being dissolved or resuspended just before use in water or an appropriate solvent (e.g., physiological saline, glucose infusion, buffering solution and the like). When the liposomes of the present invention are used as a contrast medium, a dose can be suitably determined so that the content of the compound in the liposomes becomes similar to that of a conventional contrast medium.

Although it is not intended to be bound by any specific theory, it is known that, in vascular diseases such as arteriosclerosis or restenosis after PTCA, vascular smooth muscle cells constituting tunica media of blood vessel abnormally proliferate and migrate into endosporium at the same time to narrow blood flow passages. Although triggers that initiate the abnormal proliferation of normal vascular smooth muscle cells have not yet been clearly elucidated, it is known that migration into endosporium and foaming of macrophages are important factors. It is reported that vascular smooth muscle cells then cause phenotype conversion (from constricted to composite type).

When the liposomes of the present invention are used, the compound serving as the defined contrast medium can be selectively taken up into the vascular smooth muscle cells abnormally proliferating under influences of foam macrophages. As a result, imaging becomes possible with high contrast between vascular smooth muscle cells of a lesion and a non-pathological site. Therefore, the contrast medium of the present invention can be suitably used particularly for MRI of vascular diseases. For example, imaging of arteriosclerotic lesion or restenosis after PTCA can be performed.

Further, as described in J. Biol. Chem., 265, 5226 (1990), for example, it is known that liposomes containing phospholipids, in particular, liposomes formed by using PC and PS, likely to accumulate on macrophages with the aid of scavenger receptors. Therefore, by using the liposomes of the present invention, the compounds of the present invention can be accumulated in a tissue or a lesion in which macrophages localize. If the liposomes of the present invention are used, the compound defined can be accumulated in macrophages in a larger amount compared with agents using a suspension or an oil emulsion according to a known technique.

Examples of tissues in which localization of macrophages is observed, which can be suitably imaged by the method of the present invention, include blood vessel, liver, spleen, air vesicle, lymph node, lymph vessel, and renal epithelium. Further, it is known that macrophages accumulate in lesions in certain classes of diseases. Examples of such diseases include tumor, arteriosclerosis, inflammation, infection and the like. Therefore, lesions of such diseases can be identified by using the liposomes of the present invention. In particular, it is known that foam macrophages, which take up a large amount of denatured LDL with the aid of scavenger receptors, accumulate in atherosclerosis lesions at an early stage (Am. J. Pathol., 103, 181 (1981); Annu. Rev. Biochem., 52, 223 (1983)). Therefore, by performing MRI after accumulation of the liposomes of the present invention in the macrophages, it is possible to identify locations of atherosclerosis lesions at an early stage, which is hardly achievable by other means.

The imaging method using the liposomes of the present invention is not particularly limited. For example, imaging can be attained by measuring change in the T1/T2 relaxation time of water in the same manner as that in imaging methods using an ordinary contrast medium for MRI. It is also possible to use the liposome as a contrast medium for scintigraphy, X-ray contrast medium, optical image formation agent, and ultrasonic contrast agent by suitably using an appropriate metal ion.

### EXAMPLES

The present invention will be explained more specifically with reference to the following examples. However, the scope of the present invention is not limited to the following examples. The compound numbers used in the following examples correspond to the numbers of the exemplified compounds mentioned above. The structures of the compounds mentioned in the examples were confirmed on the basis of NMR spectra and mass spectra.

### Preparation Example 1

### Preparation of Compound 2-1

(1) Palmitoleic acid (5.23 g) and 1-bromo-2,3-dihydroxypropane (1.46 g) were dissolved in dichloromethane (25 ml), and added with N,N-dimethylaminopyridine (55 mg) and ethyl-N,N-dimethylaminopropylcarbodiimide hydrochloride (4.30 g), and the mixture was stirred at room temperature for 1 day. The solvent was evaporated, and the obtained residue was purified by silica gel column chromatography to obtain 5.74 (97%) g of diacylated compound of 1-bromo-2,3-dihydroxypropane.

¹H-NMR (400 MHz, CDCl₃) δ: 5.40-5.29 (4H, m) 5.21 (1H, quin) 4.34 (1H, dd) 4.23 (1H, dd) 3.53 (1H, dd) 3.48 (1H, dd) 2.38-2.29 (4H, m) 2.05-1.95 (8H, m) 1.68-1.55 (4H, m) 1.39-1.21 (32H, m) 0.89 (6H, t).

(2) The diester compound (3.77 g) obtained in (1) was dissolved in dioxane (30 ml), and added with N,N′-dimethylethylenediamine (5.34 g), and the mixture was stirred at 90° C. for 6 hours. This solution was added with dichloromethane and saturated aqueous sodium hydrogencarbonate, and then extracted twice with dichloromethane, and the organic layer was washed with water and saturated brine. The obtained organic layer was dried over sodium sulfate, and then the solvent was evaporated. The residue was purified by silica gel column chromatography to obtain a 2.29 g of 3-amino-1,2-dihydroxypropane derivative (60%).

¹H-NMR (400 MHz, CDCl₃) δ: 5.40-5.29 (4H, m) 5.18 (1H, dq) 4.36 (1H, dd) 4.09 (1H, dd) 2.61 (2H, t) 2.55-2.50 (4H, m) 2.42 (3H, s) 2.30 (4H, dt) 2.27 (3H, s) 2.05-1.93 (8H, m) 1.68-1.52 (4H, m) 1.38-1.22 (32H, m) 0.89 (6H, t).

(3) The propanediol derivative (0.38 g) obtained in (2) was dissolved in chloroform (50 ml), and added with diethylenetriaminepentaacetic acid dianhydride (1.07 g), and the mixture was stirred at 60° C. for 30 minutes. Subsequently, the mixture was added with triethylamine (0.84 ml), and further stirred at 60° C. for 4 hours. The insoluble matter was removed by filtration, and the filtrate was added with 1 N hydrochloric acid, and extracted twice with dichloromethane. The obtained organic layer was dried over sodium sulfate, and the solvent was evaporated to obtain 0.50 g of Compound 2-1 (83%).

### Compound 2-1

¹H-NMR (400 MHz, CD₃OD) δ: 5.59 (1H, bs) 5.42-5.37 (4H, m) 4.58-4.51 (1H, m) 4.35-4.26 (1H, m) 3.98-3.38 (18H, m) 3.30-3.21 (4H, m) 3.09 (3H, s) 3.01 (3H, s) 2.50-2.35 (4H, m) 1.73-1.58 (4H, m) 1.48-1.25 (32H, m) 0.99-0.92 (6H, m).

Mass (MALDI-TOFF): m/z (α-cyano-4-hydroxycinnamic acid) 1008 (M-H)

(4) Gadolinium(III) chloride (81 mg) was dissolved in 1.5 ml of methanol, and added dropwise with a solution of Compound 2-1 (0.28 g) dissolved in methanol (3 ml). The mixture was added with 1 ml of water, and then adjusted to pH 6 with 1 N aqueous sodium hydroxide. The produced crystals were separated by filtration, and dried to obtain 0.17 g (51%) of gadolinium complex of Compound 2-1.

### Test Example 1

### Solubility Test

Each of the gadolinium complex of Compound 2-1 and Comparative Compound 1 as a known compound was weighed in an amount giving a concentration of 1 mM, and added with 1 ml of each solvent, and solubility in each solvent was examined (at room temperature of 25° C.). The results are shown in Table 1. From the results shown in Table 1, it can be understood that the solubility of the gadolinium complex of Compound 2-1 according to the present invention is improved compared with Comparative Compound 1, especially in dichloromethane and chloroform, and thus the complex has superior property for preparation of liposomes.

TABLE 1

| | Dichloromethane | Chloroform | Chloroform/methanol (1/1) |
|---|---|---|---|
| Gadolinium | ◯ | ◯ | ◯ |
| complex of | | | |
| Compound 2-1 | | | |
| Comparative | X | X | ◯ |
| Compound 1 | | | |

| | |
|---|---|
| ◯: Uniform solution was formed, | |
| X: Other observation | |
| | (Comparative Compound 1) |
| 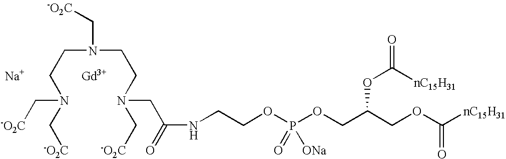 | |

### Test Example 2

### Preparation Method of Liposome

According to the method described in J. Med. Chem., 25 (12), 1500 (1982), dipalmitoyl-PC (Funakoshi, No. 1201-41-0225), dipalmitoyl-PS (Funakoshi, No. 1201-42-0237), and the compound of the present invention, in the ratio described blow, were dissolved in chloroform contained in an eggplant-shaped flask to form a uniform solution, and then the solvent was evaporated under reduced pressure to form a thin membrane on the bottom of the flask. The thin membrane was dried in vacuo, then added with an appropriate volume of 0.9% physiological saline (Hikari Pharmaceutical, No. 512) and ultrasonicated (probe type oscillator, Branson, No. 3542, 0.1 mW) for 5 minute with ice cooling to obtain a uniform liposome dispersion. Size of the particles contained in the resulting dispersion was measured by using WBC analyzer (Nihon Kohden, A-1042). The particle size was 40 to 65 nm.

PC 45 nmol+PS 5 nmol+Gadolinium complex of Compound 2-1 10 nmol

### Test Example 3

### Toxicity Test by Continuous Administration for 3 Days in Mice

Six-week old ICR male mice (Charles River Japan) were purchased, and after quarantine for 1 week, acclimatized for 1 week in a clean animal cage (air-conditioning: HEPA filter of class 1000, room temperature: 20 to 24° C., humidity: 35 to 60%). Then, in order to obtain the MTD value, a suspension of the test compound was administered from the caudal vein. The suspension was administered by using physiological saline (Hikari Pharmaceutical) or a glucose solution (Otsuka Pharmaceutical) as a medium. On the basis of the MTD value obtained, the suspension was administered everyday from the caudal vein for three consecutive days in an amount corresponding to ½ of the MTD value (n=3). Symptoms were observed up to 6 hours after each administration to observe neurotoxicity, and then autopsy was performed to examine major organs. It is clearly understood that the compound of the present invention has low toxicity and no neurotoxicity, and thus the compound has superior features as a component lipid of liposomes for a contrast medium for MRI.

Compound (MTD (mg/kg)): neurotoxicity (“−” indicates negative for neurotoxicity, and “+” indicate positive for neurotoxicity)
Gadolinium Complex of Compound 2-1 (100 mg/kg):

### INDUSTRIAL APPLICABILITY

The compounds or the chelate compounds, or the salts thereof according to the present invention have high solubility, and therefore said substances are highly suitable as a component lipid of a liposome contrast medium. A lesion of a vessel can be selectively contrasted by performing MRI or scintigraphy imaging by using the liposomes comprising said substance.

1. A compound represented by the following general formula (I), or a salt thereof: 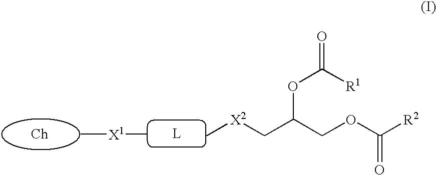
wherein R¹ and R² represent an alkyl group or alkenyl group having 8 to 30 carbon atoms; X¹ and X² independently represent —O—, or —N(Z)-; Z represents a hydrogen atom, or an alkyl group having 1 to 5 carbon atoms; L represents a divalent bridging group comprising one or more partial structures selected from the group consisting of an alkylene group, an alkenylene group, —CO—, —O—, —NH—, —N═, —S—, —SO—, and —SO₂, wherein the number of atoms that constitute the chain length of the bridging group is 1 to 10, and wherein said bridging group may be substituted; and Ch represents a partial structure containing 3 or more nitrogen atoms and capable of forming a chelate, which may be substituted with a substituent selected from the following substituent group (substituent group: a hydroxyl group; an alkoxyl group; a carboxyl group; a carbamoyl group; an amino group; an alkylamino group; a sulfo group; an alkyl group having 1 to 5 carbon atoms which may be substituted with a substituent selected from the group consisting of a hydroxyl group, an alkoxyl group, a carboxyl group, a carbamoyl group, an amino group, an alkylamino group, and a sulfo group; and a combination of these groups, provided that the total number of carbon atoms, oxygen atoms, nitrogen atoms, and sulfur atoms in each substituent in the substituent group is 1 to 20).

2. The compound or a salt thereof according to claim 1, wherein Ch is represented by the following general formula (II): 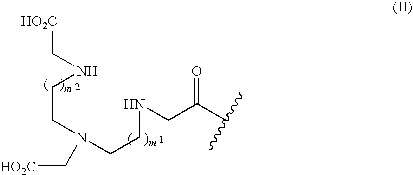
wherein m¹ and m² independently represent an integer of 1 or 2.

3. The compound or a salt thereof according to claim 1, wherein Ch is represented by the following formula (III):

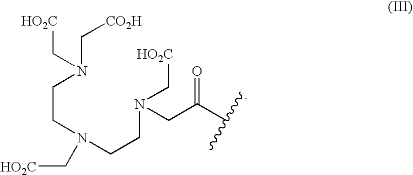

4. The compound or a salt thereof according to claim 1, wherein Ch is represented by the following formula (IV):

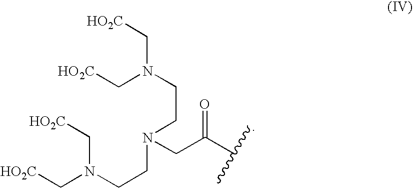

5. The compound or a salt thereof according to claim 1, wherein Ch is represented by the following general formula (V): 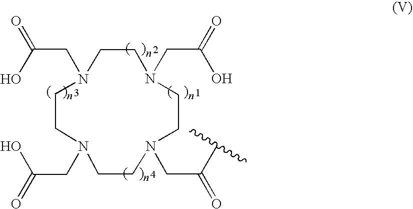
wherein n¹, n², n³, and n⁴ independently represent an integer of 1 or 2.

6. The compound or a salt thereof according to claim 1, wherein R¹ and R² independently represent an alkyl group having 8 to 30 carbon atoms.

7. The compound or a salt thereof according to claim 1, wherein R¹ and R² independently represent an alkenyl group having 8 to 30 carbon atoms.

8. The compound or a salt thereof according to claim 1, wherein R¹ and R² independently represent an alkyl group or alkenyl group having 10 to 20 carbon atoms.

9. The compound or a salt thereof according to claim 1, wherein L is a divalent bridging group comprising one or more partial structures selected from the group consisting of an alkylene group, an alkenylene group, —CO— and —O— wherein the number of atoms constituting the chain length of the bridging group is 1 to 10.

10. A chelate compound or a salt thereof, which consists of the compound according to claim 1 and a metal ion.

11. The chelate compound or a salt thereof according to claim 10, wherein the metal ion is derived from an element selected from elements of atomic numbers 21 to 29, 31, 32, 37 to 39, 42 to 44, 49, and 57 to 83.

12. The chelate compound or a salt thereof according to claim 10, wherein the metal ion is derived from a paramagnetic element selected from elements of atomic numbers 21 to 29, 42, 44, and 57 to 71.

13. A liposome containing as a membrane component the compound or a salt thereof according to claim 1.

14. The liposome according to claim 13, which contains a phosphatidylcholine and a phosphatidylserine as membrane components.

15. A contrast medium for MRI, which comprises the liposome according to claim 13.

16. The contrast medium for MRI according to claim 15, which is used for imaging a vascular disease.

17. The contrast medium for MRI according to claim 15, which is used for imaging of vascular smooth muscle cells which abnormally proliferate under influence of foam macrophages.

18. The contrast medium for MRI according to claim 15, which is used for imaging of a tissue or a lesion in which macrophages localize.

19. The contrast medium for MRI according to claim 15, wherein the tissue in which macrophages localize is selected from the group consisting of tissues of liver, spleen, air vesicle, lymph node, lymph vessel, and renal epithelium.

20. The contrast medium for MRI according to claim 15, wherein the lesion in which macrophages localize is selected from the group consisting of lesions of tumor, inflammation, and infection.

21. A contrast medium for scintigraphy, which comprises the liposome according to claim 13.

22. The contrast medium for scintigraphy according to claim 21, which is used for imaging a vascular disease.

23. The contrast medium for scintigraphy according to claim 21, which is used for imaging of vascular smooth muscle cells which abnormally proliferate under influence of foam macrophages.

24. The contrast medium for scintigraphy according to claim 21, which is used for imaging of a tissue or a lesion in which macrophages localize.

25. The contrast medium for scintigraphy according to claim 21, wherein the tissue in which macrophages localize is selected from the group consisting of tissues of liver, spleen, air vesicle, lymph node, lymph vessel, and renal epithelium.

26. The contrast medium for scintigraphy according to claim 21, wherein the lesion in which macrophages localize is selected from the group consisting of lesions of tumor, inflammation, and infection.
